(19) United States
(12) Patent Application Publication
(10) Pub. No.: US 2025/0057596 A1
(43) Pub. Date: Feb. 20, 2025

(54) MEDICAL INSTRUMENT AND SYSTEM COMPRISING SUCH AN INSTRUMENT

(71) Applicant: Bruno ANASTASIE, Saint Pierre du Perray (FR)

(72) Inventor: Bruno ANASTASIE, Saint Pierre du Perray (FR)

(21) Appl. No.: 18/721,542

(22) PCT Filed: Dec. 19, 2022
(86) PCT No.: PCT/EP2022/086682

§ 371 Date: Aug. 8, 2024

(30) Foreign Application Priority Data
Dec. 21, 2021 (FR) FR2114199

(51) Int. Cl. A61B 18/24; A61B 18/00; A61B 18/20; A61B 18/22; G01N 21/25; G01N 33/02

(52) U.S. Cl. CPC A61B 18/24; A61B 2018/00404; A61B 2018/00589; A61B 2018/00678; A61B 2018/00821; A61B 2018/00898; A61B 2018/2005; A61B 2018/2065; A61B 2018/2227; A61B 2018/2261; A61B 2018/2272; A61B 2560/0223; G01N 21/255; G01N 33/02

(57) ABSTRACT

The present invention relates to a medical instrument comprising a free distal end, intended to penetrate tissue, and a proximal end connectable to a light source, the instrument comprising a tubular support in which there is mounted at least one light guide for substantially frontal emission of the light by the guide via the first optical outlet, the instrument being characterized in that it comprises at least one means of optical deviation of the light, forming a second optical outlet, for deviated emission of light, comprising at least one machining operation on a distal portion of the instrument, with at least one reflective and/or concave polishing configured for a convergence of the light and a reflective and/or convex polishing configured for a divergence of the light, the polishing operations being carried out on the light guide and/or on the support.

### TECHNICAL FIELD OF THE INVENTION

The present invention generally relates to the field of light treatment instruments, in particular an endovenous laser that can be used to treat varicose veins. This technology can be applied in industrial environments particularly food, dentistry and veterinary medicine. In addition, the present invention is not limited to endovenous treatment and may relate to various types of surgical procedures, such as conization or any other procedure requiring a tool enabling both detersion and/or destruction and photocoagulation. Similarly, the present invention provides a novel solution for lasers and CO2 lasers in particular, but is not limited to lasers and covers various types of light, at various wavelengths.

### STATE OF THE PRIOR ART

Surgical operations requiring detersion and/or destruction are often costly and time-consuming, not least because they generally require general anesthesia. Further, they generate scars, particularly due to the fact that it is not always easy for the tissues to coagulate. In this context, it is interesting to propose a tool that facilitates detersion and/or destruction and improves photocoagulation possibilities.

More recent techniques for vascular sclerosis, in particular the treatment of varicose veins, such as the technique disclosed in US 2003/0078569, are less burdensome for the patient, but nevertheless require catheterization preparation, dressing of the operator, sterile drapes, extensive disinfection and complex handling in the operating room. They further use long and expensive optical fibers. In addition, this technique requires the injection of anesthetic products that can have undesirable effects, for example allergies. This is the case for conventional sclerosis in a medical practice, which uses chemical sclerosants.

US 2005/0131400 details a device for conducting a laser in an optical fiber, with the end of the optical fiber passing through a catheter to be inserted into a blood vessel. The laser beam scattered at the end of the optical fiber inside a vein helps to treat varicose veins by acting on the endothelial cells of the vein wall as well as on the entire vein wall, the effect being thermomechanical. However, such a device does not optimize light diffusion during circumferential treatment of the wall of a vessel.

Application WO2007104836A1 teaches a tubular support such as a needle in which a light guide such as an optical fiber is present, and discloses the use of lateral windows in the support or various arrangements of the distal end of the instrument for light scattering. However, this type of instrument can be improved, particularly in terms of light deviation, by convergence or divergence, depending on the type of target application.

The aim of the invention is to propose a more simple technique and an equipment, instrument and system that are less costly and easier to handle and/or that improve the possibilities of detersion, tissue destruction and photocoagulation.

### DISCLOSURE OF THE INVENTION

The object of the present invention is therefore to propose a medical instrument and a system comprising such an instrument, making it possible to overcome at least some of the disadvantages of the prior art by proposing a more simple technique and an equipment, instrument and system, which is less costly and easier to handle and/or which improves the possibilities of detersion, tissue destruction and photocoagulation. One of the advantages is thus to reduce the cost of the procedure outside the operating theater and to be able to repeat the operation at lower cost if necessary.

This aim is achieved by a medical instrument comprising a free distal end, intended to penetrate a tissue or organ and/or a blood vessel and a proximal end connectable to a light source, said instrument comprising a tubular support in which there is mounted at least one light guide, said support comprising a beveled distal end comprising at least one orifice forming a first optical outlet, for substantially frontal emission of the light by said guide via said first optical outlet, said instrument being characterized in that it comprises at least one means of optical deviation of the light, forming a second optical outlet, for deviated emission of light, comprising at least one machining operation on a distal portion of the instrument, with at least one reflective and/or concave polishing configured for a convergence of the light and a reflective and/or convex polishing configured for a divergence of the light, said polishing operations being carried out on said light guide and/or on said support.

According to another feature, the light guide comprises an optical fiber.

According to another feature, said optical fiber is hollow and associated with a CO2 laser light source.

According to another feature, the light guide comprises a reflective coating on the inner wall of the tubular support.

According to another feature, said beveled distal end of said support is arrow-sharpened at a sharpening angle imparting cutting and penetrating properties.

According to another feature, at least one distal portion is curved according to a radius of curvature, facilitating manipulation of the instrument from outside the tissue or organ.

According to another feature, said light deviation means comprises at least one lateral orifice in the wall of said support, near the distal end, said lateral orifice comprising at least one chamfer in the thickness of the wall of the support, providing an angle of optical deviation, either flaring outwards to form a divergent deviation means, or flaring inwards to form a convergent deviation means.

According to another feature, said light deviation means is a divergent deviation means comprising a convex polishing carried out on a light-conducting ball deposited on the surface of the light guide.

According to another feature, the distal end of the light guide, for example a cross-sectioned or beveled bare-ended optical fiber or a hollow fiber, is positioned set back from the beveled distal end of the support, with a reflective polished inner surface inside the beveled distal end of the support then serving as a reflective tool forming a means of optical deviation.

According to another feature, the beveled distal end of the support has a circumferential polishing area on the surface of the bevel at a bevel angle and comprises a second bevel, obtained by a second polishing of a portion of said polishing area.

According to another feature, said second polishing is carried out on a lateral portion of said polishing area, providing a lateral optical deviation angle.

According to another feature, said second polishing is circumferential over said entire polishing area and provides a distal optical deviation angle.

According to another feature, said second polishing is carried out on a central and apical portion of said polishing area, providing an apical optical deviation angle.

According to another feature, said second polishing is performed at a height determined to also obtain a polishing area of the distal end of the light guide, resulting in two angles of optical deviation and a deformed active surface of the light guide.

According to another feature, the beveled distal end of the support comprises a circumferential polishing area on the surface of the bevel at a bevel angle and comprises at least one semi-spherical or triangular indentation forming at least one means of optical deviation, either lateral, or central and apical.

According to another feature, the distal end of the light guide is housed inside the support, set back from the distal end of the support by a distance of at least 1 to 10 mm.

According to another feature, the instrument comprises two light guides, preferentially two optical fibers, one of the guides carrying a means of divergent optical deviation, for example convex polishing, while the other carries a means of convergent optical deviation, for example concave polishing.

According to another feature, said light guide comprises both a means of divergent optical deviation and a means of convergent optical deviation.

According to another feature, the support comprises both a means of divergent optical deviation and a means of convergent optical deviation.

According to another feature, the instrument comprises means for rotating and/or advancing the light guide and/or the support, in particular relative to each other.

According to another feature, the instrument comprises at least one marking to check the correct positioning of the instrument.

According to another feature, at least one colored filter is placed in the path of the light.

According to another feature, the support is made of a biologically neutral material, preferably selected from stainless steel or aluminum.

According to another feature, the support has a distal coating of a material with enhanced heat conduction.

According to another feature, the light guide is an optical fiber reflecting wavelengths between 200 nm and 5 μm.

According to another feature, the light guide is a coating of the inner surface of the support forming an inner reflective tube reflecting wavelengths between 200 nm and 11 μm, preferably 5 μm and 11 μm.

According to another feature, the light guide is a doped optical fiber with an outer diameter of between 50 and 1000 μm, preferably between 50 and 150 μm, preferably between 75 and 125 μm.

According to another feature, the support has a length of between 10 and 120 mm, the outer diameter of the guide is between 100 and 4000 μm, preferably 50 and 1000 μm, the outer diameter of the needle is between 200 and 5000 μm, preferably between 450 μm and 1000 μm and the distal end of the support is beveled with a bevel angle of between 1° and 20°.

According to another feature, it comprises an electronic accounting device with the light vector and the source in order to calibrate the power of the beam at the needle outlet.

According to another feature, the guide aperture, preferably the numerical aperture of the fiber, is between 15° and 40°, preferably 20° and 35°, still preferentially 23° and 32°.

According to another feature, the instrument comprises two channels, either a double channel in the support, or a channel in the support associated with another channel of another instrument, the assembly being coupled to a thermocouple measuring device for measuring the temperature at the distal end of the support.

According to another feature, the instrument further comprises an alarm system when the measured temperature value exceeds a predetermined threshold value.

According to another feature, the instrument further comprises a thermal reagent configured to react when said instrument reaches a predetermined temperature, in order to visualize overheating.

According to another feature, the light guide comprises a plurality of different optical fibers, with different means of optical deviation from one fiber to another, the instrument being coupled to a multiplexer at the light source to perform photodiagnostics thanks to the differential collection of signals from the different fibers.

Another purpose of the invention is to provide a food control process that is easy and inexpensive to implement.

This purpose is achieved by a process for detecting toxic products in food control using a medical instrument according to the present application, by detecting chromophores.

### BRIEF DESCRIPTION OF THE FIGURES

Further features, details and advantages of the invention will become apparent from reading the following description with reference to the appended figures, which show:

FIG. 1 shows a schematic view of a system according to certain embodiments of the invention, in use;

FIG. 2 shows a schematic profile view of the connection means of the system according to some embodiments;

FIG. 3 is a longitudinal cross-sectional view of the connection means shown in FIG. 2;

FIG. 4 is a schematic profile view of the connection means and the tubular support according to some embodiments;

FIG. 5 is a schematic view of the distal bevel of the tubular support, according to some embodiments;

FIG. 6 is a schematic perspective view of the distal end of the support, according to some embodiments comprising a second lateral distal bevel;

FIG. 7 is a schematic perspective view of the distal end of the support as shown in FIG. 6, and further showing the light guide and the digital aperture thereof according to some embodiments;

FIG. 8 is a schematic perspective view of a tubular support and the light guide of an instrument according to some embodiments comprising a second circumferential distal bevel;

FIG. 9 is a schematic transparent perspective view of the support and the light guide formed by a hollow tube for conveying a CO2 laser of an instrument according to certain embodiments;

FIG. 10 is a schematic profile view of the support with distal curvature according to some embodiments;

FIG. 11, FIG. 12 and FIG. 13 show schematic perspective details of the distal end of the support in various embodiments comprising at least one lateral distal window with polishing;

FIG. 14 is a schematic perspective view of a tubular support and the light guide of an instrument according to some embodiments comprising a second central and apical distal bevel;

FIG. 15 is a schematic front view of the distal end of an instrument according to some embodiments comprising a second central and apical distal bevel which extends beyond the light guide and thus forms a polishing of the distal end of the light guide;

FIG. 16 is a schematic front view of the distal end of an instrument such as that shown in FIG. 15, showing two different heights of the central and apical distal second bevel extending beyond the light guide, with the active surface of the light guide deformed by its distal polishing;

FIG. 17 is a schematic perspective view of the distal end of an instrument such as that shown in FIG. 16, showing the angles of the distal bevel on the light guide and the tubular support and the deformation of the active surface of the light guide;

FIG. 18 is a schematic perspective view of a tubular support and the light guide of an instrument according to some embodiments comprising at least one lateral, semi-spherical and/or triangular distal indentation.

### DETAILED DESCRIPTION OF THE INVENTION

The present application relates to a surgical instrument for light treatment, in particular laser treatment. Numerous combinations of the embodiments detailed in the present application can be envisaged without departing from the scope of the invention; the person skilled in the art will select one or other according to the economic, ergonomic, dimensional or other constraints that said person must comply with in line the regulations. In addition, the various technical features are described in functional terms and it is understood from reading the present application that a single device may combine the functional features of various embodiments.

Light treatment is well known in the medical field and particularly laser treatment. As a reminder, the effects of this treatment can be both thermal and mechanical. Thermal effects occur when laser radiation is absorbed by the obstacle (the tissue). They then induce a tissue reaction that is linked to changes in body temperature and the duration of heating. Depending on the temperature increase in the tissue, different types of reaction can occur.

Hyperthermia corresponds to a moderate increase in tissue temperature, of around a few degrees. Thus, a tissue temperature of around 41° C. for several tens of minutes can lead to cell death. Coagulation corresponds to irreversible necrosis without immediate tissue destruction. In this action, the temperature of the tissue can reach temperatures of between 50° C. and 100° C. for about one second. This temperature then produces desiccation, whitening and retraction of tissues through denaturation of proteins and collagen. The tissues will then be eliminated (detersion) and heal. Volatilization or vaporization corresponds to a loss of substance. Herein, we're talking about tissue temperatures in excess of 100° C. Under these conditions, cell components are evaporated over a relatively short period of time. A zone of coagulation necrosis can be seen at the edges of the volatilized zone as the thermal transition between the volatilized zone and the healthy zone occurs gradually. Mechanical effects are induced by the creation of a plasma, explosive vaporization or by cavitation. These effects are mainly linked to the expansion of a shock wave (created from thermal effects) which will generate a destructive effect. In fact, when material is ejected from the substrate by illumination, said substrate will recoil. This recoil effect is linked to energy conservation and the fact that light energy is converted into kinetic energy. The present application proposes to take advantage of these effects in order to optimize treatment options.

According to a first objective of the invention, such an instrument comprises a light guide, for example an optical fiber, and a rigid tubular support accommodating the guide, for example a needle forming a protective and guiding sheath around the light guide. The terms “tubular support” and “needle” are therefore used in this application in a non-limiting manner and interchangeably. Similarly, the terms “light guide”, “light vector”, “vector”, “optical fiber” or “fiber” are used in the present application in a non-limiting manner and interchangeably.

Such an optical needle is connected to a laser or white light source, for example filtered, of variable wavelength by virtue of the light vector contained therein and which conveys the light from the source to the distal end. The connection (4) to the light source can be achieved via a standard connector (SMA or other), which can be re-sterilized (particularly chemically) for use in sterile surgical environments. Thus, in contrast to previous uses where the flexibility of an optical fiber is preferred, in order for example to be able to traverse (catheterize) a vein or even an artery over a long distance from an entry of the fiber up to an area to be treated, on the contrary, a certain rigidity is preferred which is provided by the support. Of course, this rigidity is not absolute, but rather should be compared with the flexibility of an optical fiber as used, particularly in endovenous lasers. This rigidity can be refined according to use. For example in the case of a beveled needle, said needle may be sufficiently rigid to enable it to penetrate the skin of a patient, cut through tissue such as a tumor lesion or deterge tissue such as an ulcer for example.

Advantageously, the instrument may comprise connection means to a light source, in particular a laser light source. The laser light source may have a wavelength of between 200 and 4000 nm, which is suitable for endovascular use. Other wavelengths are also possible depending on the application. In particular, embodiments are provided wherein a hollow fiber (or reflective coating) is used in the hollow needle, with internal reflection, especially for a CO2 laser (10.6 μm wavelength making it impossible to use a solid optical fiber).

In some embodiments, the laser light source may have a wavelength advantageously between 800 and 1000 nm. Even more advantageously, this wavelength is 980 nm, due to its preferential absorption by oxygenated hemoglobin and water.

The instrument comprises means for substantially frontal light emission and means of light deviation, in particular for lateral light emission or deviation not parallel to the frontal emission axis (corresponding to the generatrix of the cylinder formed by the needle and/or the fiber). Lateral diffusion can take place through one or more lateral windows and/or by means of a lateral bevel on the distal end of the instrument and/or by means of a polishing (or circumferential or apical bevel or flat), either on the support, or on the guide, or on both. Depending on the intended use, the windows may be distributed transversely or longitudinally along the light guide and/or the support. In some embodiments, the type of polishing used is called “gloss” polishing. In other embodiments, the type of polishing used is “matt” polishing. This type of polishing is more costly but allows light to be channeled in a desired direction.

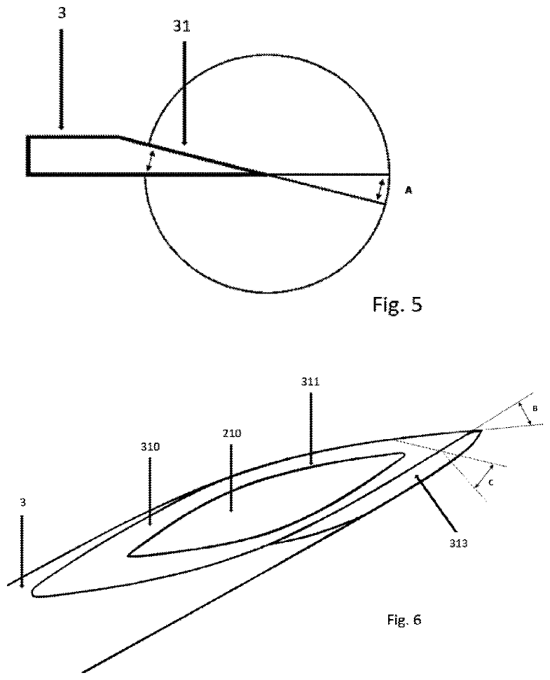

Generally speaking, the present invention comprises a medical instrument (1) comprising a manipulable free distal end intended to penetrate a tissue or organ (6) and/or a blood vessel (V) and a proximal end connectable to a light source (10), said instrument comprising a tubular support (3) in which there is mounted at least one light guide (2). This support (3) comprises a beveled distal end (31) comprising at least one orifice forming a first optical outlet, for substantially frontal emission of light by said guide (2) via said first optical outlet. This beveled distal end is arranged to facilitate penetration of the needle, as known in the prior art. The angle (A) of this distal bevel may vary according to the size of the needle, particularly in diameter or length, as shown for example in FIG. 5. The bevel angle of the needle is between 10-40°, preferably 20° and 30°, still preferentially 25°. The more the angle is reduced, the less painful the penetration through the skin; this angle therefore conditions the algogenic nature of the puncture. It also conditions the shape of the photon field: the optical surface and active surface dependent on the numerical aperture of the light guide (2), herein the fiber, are increased when the bevel angle is smaller. The bevel angle optically affects the shape of the photon field, and therefore the biological effect.

According to various embodiments of the invention, the instrument comprises at least one means of optical deviation (210, 310, 312, 313, 314, 315) of the light forming a second optical outlet, for deviated emission of light. This means of deviation, which can take various forms according to a variety of embodiments, comprises at least one machining and/or polishing operation on a distal portion of the instrument. The polishing operation can be reflective for straight deviation of the light and/or concave for light convergence and/or convex for light divergence. Advantageously, this polishing operation is carried out on said light guide (2) and/or on said support (3). In the case of reflective polishing inducing a right deviation of the light, depending on the position of this reflective polishing on the guide (2) and/or the support (3), this deviation can either extend the active zone and thus generate a divergence of the light, or focus part of the active zone and thus generate a convergence of the light. It is understood that several different polishing operations can be carried out at different locations on the distal portion of the instrument, in order to obtain several means of light deviation, which make it possible to combine the effects produced by each, in one and the same tool, thus minimizing the costs and invasiveness of the medical device. Indeed, light convergence, for example through concave polishing, promotes tissue detersion, particularly sectioning, and even destruction. Conversely, light divergence, for example through convex polishing, promotes photocoagulation and/or photodiagnosis and/or photodynamic therapy. For example, a right reflective polishing on the inner wall of a beveled end of the light guide will enable part of the light to be focused on the part of the bevel that is not provided with this reflective polishing and thus produce a convergence allowing tissue detersion, sectioning or destruction. On the other hand, depending on the numerical aperture of the light guide and the angle of the distal bevel, it is possible to allow light divergence through reflective polishing, for example when the guide is set back in the support provided with such polishing at the distal end thereof. Similarly, polishing to form a second bevel or a flat or a chamfer at the edge of an outlet orifice will allow light to diverge, thereby promoting photocoagulation. Various configurations combining means of convergent light deviation with divergent deviation means are therefore envisaged in the present application, on the light guide and/or the support, to obtain a single tool combining the therapeutic effects of the convergent light and divergent light. It is also worth noting that various embodiments offer greater reliability and solidity thanks to the protection offered by the support and the stability conferred by the machining operations of the tool. Thus, rather than using a convex polishing obtained by a ball deposited at the distal end (by the technique known as “ball fiber”) which is costly and complex to implement, it is possible to machine the support and/or the guide in order to obtain a divergence of the light at lower cost, while controlling the extent of diffusion based on the angle (C, G, D, F) of the bevel, flat or chamfer and its polishing, as detailed below. Thus, instead of using a single bevel or distal polishing as in the prior art, various embodiments advantageously use a second polishing (or bevel), either of the support, or of the fiber, or of both. Other embodiments advantageously use a second polishing of a coating inside the support. The term “substantially frontal emission” means light emission at the distal end that is frontal to the optical outlet, but it is understood that, due to the presence of the distal bevel, this emission is not perfectly frontal and that the light is emitted in a direction that is not parallel to the generatrix of the cylinder formed by the support and/or the light guide.

Generally speaking, the instrument may comprise an optical fiber as light guide (2), for example a fiber of a type commonly used for medical applications, for example made of silica, particularly straight silica. This fiber can be mounted in a tubular support (3) such as a needle. “Needle” is understood to mean a tubular structure or support, sufficiently rigid to allow precise manipulation of the light guide. Additionally, particularly for endovenous treatment, this needle must be sufficiently rigid to pierce the skin, such as a puncture needle. It is understood that the instrument has a first end, referred to as distal in the present application, which is intended to penetrate tissue and another end which is intended to be connected to a light source (particularly a laser). The tubular support forms a cylinder between these two ends and defines a generatrix of the cylinder, which may be rectilinear, but sometimes curved as shown in FIG. 10, for example. The radius (E) of curvature of the instrument may vary depending on the application (in particular depending on the depth at which it is desired to work in the tissue) as it allows the instrument to be manipulated from outside the tissue or organ. The term “distal” therefore refers to the free end of the instrument that penetrates the tissues, organ (6) or blood vessels (V), and the term “apical” refers to the apex, that is, the furthest point or zone on this distal end. On the other hand, the instrument has another end, called the proximal end (because it is closest to the user), which can be connected to a light source to illuminate the light guide of the instrument. This proximal end is also connectable to other devices according to various embodiments detailed in the present application.

In some embodiments, the diffusion orifices of the support and the light guide, particularly the light deviation means, are located close to a distal portion (31) of the instrument of the tubular support, either at their apical end and/or at least one lateral window allowing lateral or circumferential treatment if the instrument is rotated.

The term “close” is understood to mean that the diffusion orifice is closer to the apical end of the component than to its base, preferably closer to the apical end of the component than to the middle of the component, for example at the apical end of the component. Thus, an orifice at the apical end of the support (and guide) allows straight treatment, while an orifice forming a lateral window allows circumferential treatment to be performed by rotating the tool.

In some embodiments, the end of the support (3) is a concave or convex semicircle, for example in the shape of a “cutting spatula”, allowing tissue detersion or incision (for example, cervical conization, ulcer detersion, tissue sectioning). This increases the sectioning effect particularly if the polishing of the optical surface is concave with a focusing action on the optical field thus concentrating the light energy or energy density. This thus increases the precision of the incision.

### Means of Light Deviation

The instrument comprises at least one means of light deviation, by virtue of at least one second bevel or flat or polishing, in addition to the beveled distal end (31) of the support (3). This polishing at the needle tip may be convex or concave in order to diffuse or focus the output beam, the aim being to increase the optical diffusion field (in the case of convex polishing) for tissue destruction (for example for ulcer detersion) or to focus it (in the case of concave polishing) for tissue sectioning (for example for conization).

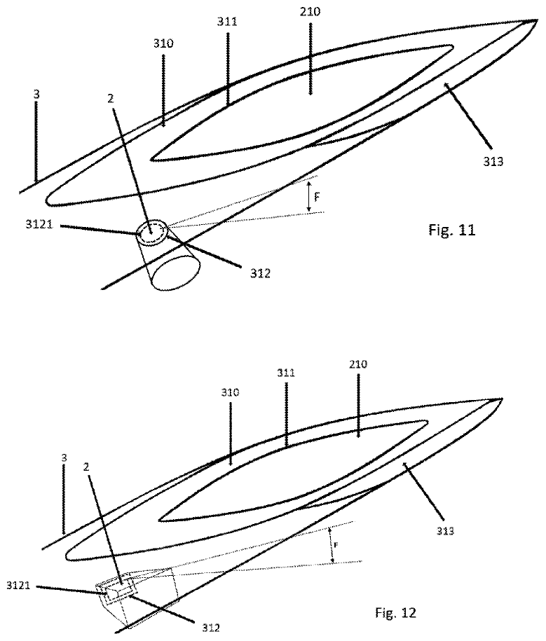

In some embodiments, said means of light deviation comprises at least one lateral orifice (312) in the wall of said support, close to the distal end (31), said lateral orifice (312) comprising at least one chamfer (3121) in the thickness of the wall of the support (3), providing an angle (F) of optical deviation, either flaring outwards to form a divergent deviation means, or flaring inwards to form a convergent deviation means. FIG. 11, FIG. 12 and FIG. 13 show illustrative and non-limiting examples of such lateral orifices which can be of various shapes and number. The chamfer allows light to be diffused in a different way to the that of the distal bevel of the instrument. For example, with a divergent angle as shown in these figures, the lateral orifices promote photocoagulation. The result is therefore a tool that cuts at its apex and coagulates in its distal portion, at lower cost. In some embodiments, such as for example shown in a non-limiting manner in FIG. 11, FIG. 12, and FIG. 13, the windowing of the support (3) (for the needle or sectioning tool) may be circular, elliptical, square or rectangular depending on the application. The number varies according to the application particularly for thermal and thermo-mechanical effects in endovenous vascular applications. There is a chamfer angle (F) which can vary depending on the machining of the device, and which in turn conditions the shape of the photon field and therefore the biological effect. It can be added at a judiciously determined position posterior or lateral to the bevel in order to add a coagulation effect and circumscribe the section above the bevel for example. The chamfer angle through the wall of the needle body is generally between 30-90°, preferably 50-70°, preferentially 60°.

Various embodiments of the present invention aim to provide an instrument with a first optical outlet, generally parallel to the generatrix of the cylinder formed by the support (3) and/or the guide (2), and a second deviated optical outlet, generally not parallel to the generatrix of the cylinder formed by the support (3) and/or the guide (2). The result is thus at least two different angulations of optical surfaces (or active surfaces) for the same instrument, notably with reduced manufacturing costs and ease of use.

In some embodiments, said means of light deviation is a divergent deviation means comprising a convex polishing carried out on a light-conducting ball deposited on the surface of the light guide (3) (by the technique known as “ball fiber”).

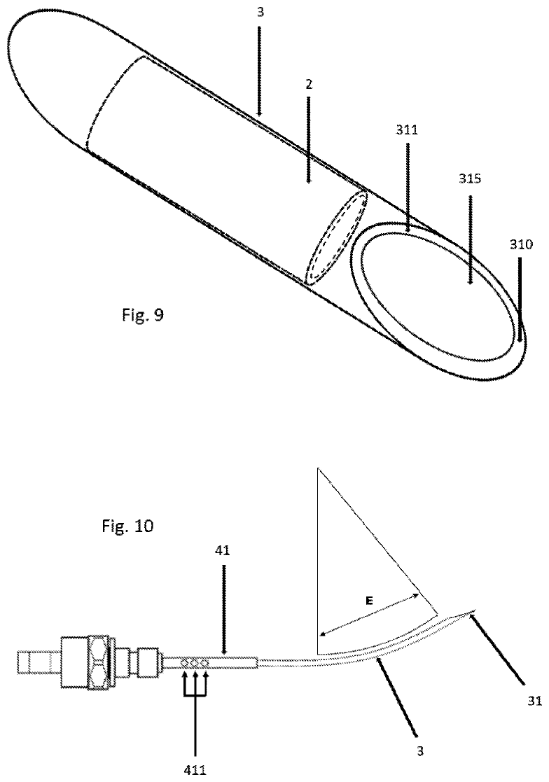

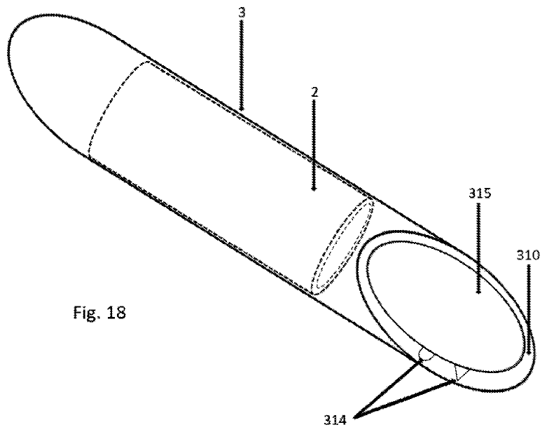

In some embodiments, the end of the light guide, for example a cross-sectioned or beveled bare-ended optical fiber or a hollow fiber, is positioned set back from the beveled distal end (31) of the support (3), with a reflective polished inner surface (315) within the beveled distal end (31) of the support (3) then serving as a reflective tool forming a means of optical deviation. This advantageously allows a less costly embodiment than when polishing is carried out. In other embodiments, a reflective device or coating is provided at the end of the needle, on the inner portion of the bevel not covered by the fiber. There are two solutions for conveying light over the beveled distal portion that extends beyond the fiber (whether hollow, for example as in FIG. 9 and FIG. 18, or otherwise). The first is to leave the metal surface uncoated to allow light to reflect as the inner face of the bevel end is already metallic and polished (generally made of biologically neutral stainless steel, as required for CE marking). This reduces the production cost of such a product. A second advantageously consists in limiting the polishing of said end of the bevel to the sharpening of this zone. This provides good value for money. In some of these embodiments, the distal end of the light guide (2) is housed inside the support (3), set back from the distal end (31) of the support by a distance, preferentially a gap of at least 1 to 10 mm. This distance may vary from one instrument to another or may be variable on the same given instrument, thanks to a translatory motion of the fiber within the needle. This variation in distance varies the photon field at the distal end of the instrument thus advantageously making it possible to select the physical effect obtained by the instrument (detersion, photocoagulation, etc.). It should also be noted that, as an alternative to a hollow fiber, a reflective covering or coating may be used inside the needle. This coating can stop at a distance from the distal end of the support (3) to obtain the same effects as for a hollow or solid fiber. Indeed, in some embodiments, the light guide (2) is not an optical fiber independent of the needle, but a covering or coating on the inside of the needle (3). This covering can be attached to the needle over the entire covered surface. Alternatively, the guide and support remain independent. It may, for example, be made of silica and offer the same light-guiding qualities as an optical fiber made of the same material, but it may also be made of a different material capable of conveying, for example, longer or shorter wavelengths. It further benefits from the protection afforded by the sheath formed by the support or needle (3), particularly in terms of strength and impact protection. Furthermore, when the covering is integral with the needle, the precision of the instrument is enhanced. In some embodiments, the light guide is an optical fiber reflecting wavelengths between 200 nm and 5 μm. Furthermore, in some embodiments, the light guide is a hollow fiber or a coating of the inner surface of the support forming an internal reflective tube reflecting wavelengths between 200 nm and 11 μm, preferably 5 μm and 11 μm. It should also be noted that these hollow-fiber or coating embodiments can be combined with the various means of deviation of the present application, whether lateral window or lateral, circumferential or apical polishing or else indentations, as detailed below.

In some embodiments, the beveled distal end (31) of the support (3) has a circumferential polishing area (310) on the surface of the bevel at a bevel angle (A) and comprises a second bevel, obtained by a second polishing (313) of a portion of said polishing area (310). This second polishing thus forms a light deviation means for deviated emission of light by the instrument. In some of these embodiments, said second polishing (313) is carried out on a lateral portion of said polishing area (310), providing an angle (C) of lateral optical deviation. The lateral polishing angle of the bevel (C) determines penetration due to the sectioning and cutting nature of the edge of the needle.

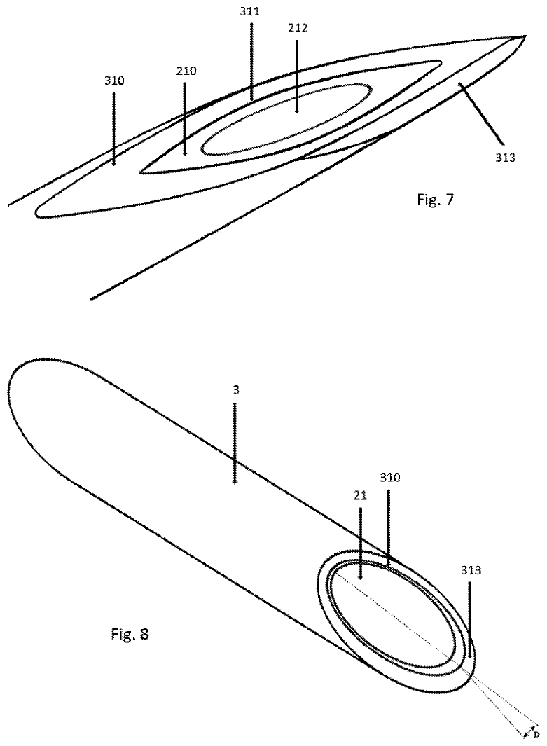

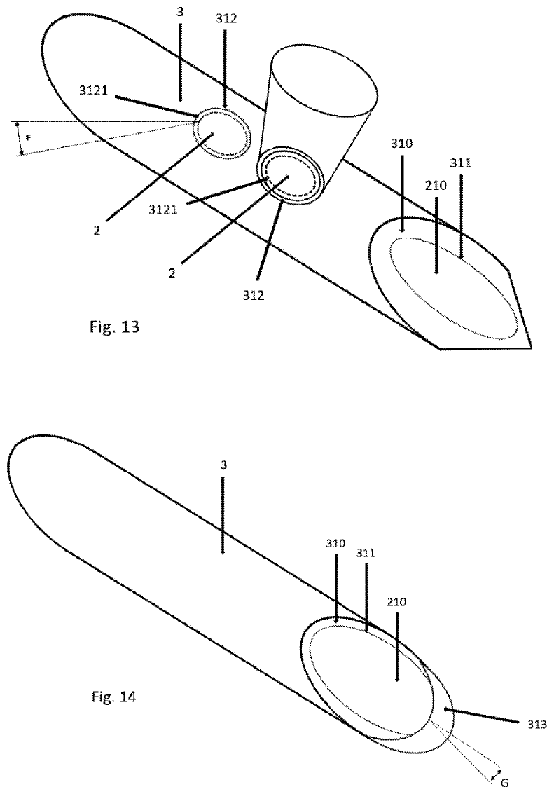

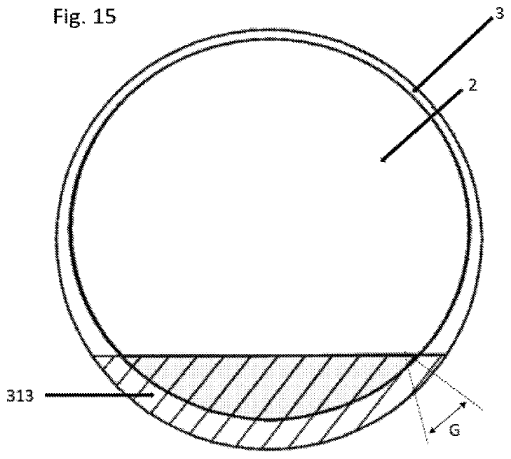

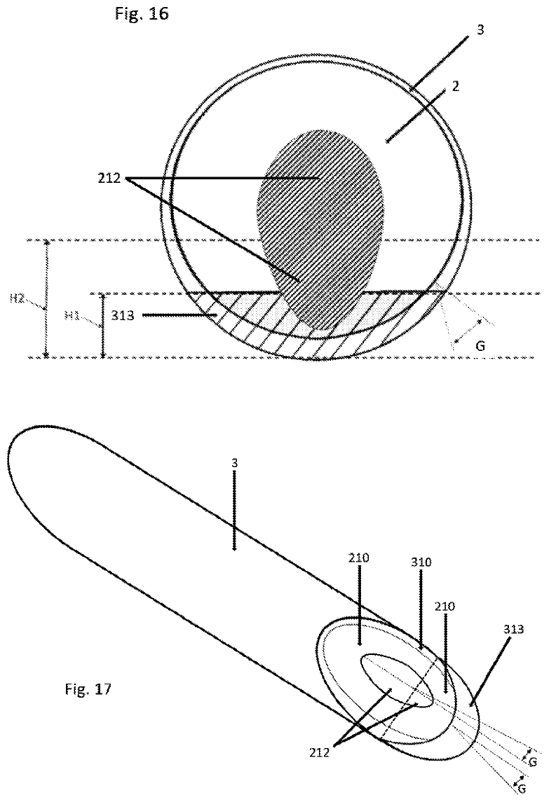

Furthermore, it also conditions the overflow of the photon field, and therefore its shape, and therefore the biological effect. Preferably, the lateral polishing angle of the bevel (C) is between 10-40°, preferably 20° and 30°, still preferentially 25°. Illustrative and non-limiting examples of such polishing or lateral bevel are shown in FIG. 6, FIG. 7, FIG. 11 and FIG. 12. Note that if this second polishing is associated with a lateral orifice, the latter is aligned relative to the second polishing, for example to obtain photocoagulation through the lateral orifice at the cross-sectional point obtained by the lateral polishing. Generally speaking, the lateral orifices are generally aligned along the generatrix of the cylinder formed by the support, so as to provide their effect in the extension of the distal bevel or means of deviation located further towards the apex than this lateral orifice. Thus, when the instrument comprises two lateral orifices, they are generally aligned with said generatrix. Furthermore, in some of these embodiments, said second polishing (313) is circumferential throughout said polishing area (310) and provides an angle (D) of distal optical deviation. The angle of distal deviation (D) will condition the photon field at its lower overflow and therefore the biological effect of sectioning strengthened by the quality of the machining of the device edge which also conditions the mechanical sectioning ability of the tool. An illustrative and non-limiting example of such polishing is shown in FIG. 8. On the other hand, in some of these embodiments, said second polishing (313) is carried out on a central and apical portion of said polishing area (310), providing an angle (G) of apical optical deviation. Illustrative and non-limiting examples of such polishing or apical bevel (or flat) are shown in FIG. 14, FIG. 15, FIG. 16 and FIG. 17. These embodiments allow for example photocoagulation to be achieved by the second apical polishing at the edges of the section obtained by the distal bevel of the support.

Furthermore, when the second apical polishing (313) is carried out on both the support and the fiber, an advantageously deformed active surface (212) is obtained, which provide for a multiple effect of the instrument on tissues, organs (6) or vessels (V). Indeed, in some of these embodiments with apical polishing, said second polishing (313) is carried out at a height (H1, H2) determined to also obtain a polishing area (210) of the distal end of the light guide (2), resulting in two angles (G) of optical deviation and a deformed active surface (212) of the light guide (2) for example as shown in FIG. 16 and FIG. 17. Finally, in some embodiments, the beveled distal end (31) of the support (3) comprises a circumferential polishing area (310) on the surface of the bevel at a bevel angle (A) and said light deviation means comprises at least one semi-spherical and/or triangular indentation (314) forming at least one means of optical deviation, either lateral, or central and apical. An illustrative and non-limiting example of such indentations is shown in FIG. 18.

### Support/Needle

In general, the support (3) is made of a biologically neutral material, preferably selected from stainless steel or aluminum. Biologically neutral materials are selected, with variable thermal conductivity to limit undesirable effects such as burning at the puncture site, and preferably resistant to high temperatures. The result is thus a biologically neutral instrument. Furthermore, thanks to the use of deviation means, the invention allows the use of powers such that the fiber is never damaged and therefore does not inoculate tissues with carcinogenic substances, unlike certain devices of the prior art. Indeed, it is important to guarantee the biological neutrality of the instrument at temperatures well above 150° C., beyond which the prior art often observes not only destruction of the fiber core, but also combustion of its sheath (polymide or tefzel, for example), generating toxic combustion products (for example polycyclic aromatic hydrocarbons recognized as carcinogens by the WHO). In addition, it is still often necessary to add a drop of glue to the needle tip, due to the manufacturing technique, but this glue is biologically neutral.

In some embodiments, it is possible to sharpen the distal end of the instrument into an arrow, for better penetration and cutting effect of the needle, for example as shown in FIG. 6, FIG. 7, FIG. 11, FIG. 12 and FIG. 13, The angle (B) of the arrow can vary, for example as shown in FIG. 6. The arrow angle (B) conditions penetration through the skin: the higher it is, the less painful the puncture. Preferably the arrow angle of the needle (B) is between 10-40, preferably 20° and 30°, still preferentially 25°. In other embodiments, the distal end is not sharpened into an arrow but remains circular or rather elliptical in shape because it is beveled for better tissue penetration. These embodiments also allow the medical instrument to be used as a detersive or sectioning tool. Thus, certain embodiments relate to an instrument wherein said beveled distal end (31) of said support (3) is arrow-sharpened to a sharpening angle (B) imparting cutting and penetrating properties.

In some embodiments, the support, for example a needle, is curved. This advantageously limits the stress at the tip of the needle and facilitates the vascular puncture procedure. As for example shown in a non-limiting manner in FIG. 10, the support (3), herein a needle, is curved. The use of a curved needle is perfectly feasible from a technical viewpoint in order to be able to reach anatomical structures during surgery in the operating theater or medical practice, particularly for phlebology, in order to optimize catheterization and the angle thereof through the vessel. The radial angle (E) can be modified to suit specific applications. It is generally between 10° and 40°, preferably between 20 and 35° and ideally between 22 and 32° for medical practice.

The needle length, needle diameter and angulation of the bevel thereof may vary according to the application. Thus, they will be larger and longer for conization and tissue (ulcer) detersion. Preferably, especially for the treatment of varicose veins, the needle may have an outer diameter in the range of 200 and 5000 μm, preferably between 450 μm and 1000 μm. The light guide may have an outer diameter of 100 to 4000 μm (4 mm), preferably between 50 and 1000 μm. Furthermore, the distal end (31) of the support (3) is preferably beveled with a bevel angle (A) of between 10 and 20°. On the other hand, the inner diameter of the support (3) may be greater than or equal to the outer diameter of the fiber (2) to accommodate any fiber sheathing and/or to form a channel around the fiber, particularly for injecting substances and/or controlling blood reflux. The instrument may further comprise a channel, with the reflux of blood in the channel attesting to the correct intravascular position of the needle; a channel can also be provided for injecting an anesthetic or adapting a thermocouple or an optical tool for spectrophotometry, or injecting a product for studying tissue fluorescence. Furthermore, in some embodiments, the needle comprises a double channel. The second channel may include a secondary fiber carrying another light for photodiagnostics, particularly in spectroscopy, but also for thermocouple measurement at the distal end of the instrument. Thus, in some embodiments, the instrument comprises two channels, either a double channel in the support (3), or a channel in the support (3) associated with another channel of another instrument, the assembly being coupled to a thermocouple measuring device for measuring the temperature at the distal end (31) of the support (3). In some of these embodiments, the instrument is connected to or further comprises an alarm system when the measured temperature value exceeds a predetermined threshold value. On the other hand, in some of these embodiments, the instrument further comprises a thermal reagent configured to react when said instrument reaches a predetermined temperature, in order to visualize overheating. In some embodiments, the support has a distal coating of a material with increased thermal conductivity, to facilitate obtaining a desired treatment temperature, in particular to amplify the thermal effect within the tissue by maintaining the proximal part of the needle with a material of lower thermal conductivity, or even thermally insulating, in order to preserve the skin.

In some embodiments, the support (3) has a length of between 10 and 120 mm, preferably with a spatulate shape. Needles are generally shorter and smaller in diameter for vascular procedures particularly in the absence of anesthesia, or for laser lipolysis.

In some embodiments, the instrument is connected to or comprises an electronic accounting device with the light vector and source in order to calibrate the power of the beam at the needle outlet. This type of technique is well known and allows the output power to be checked, particularly advantageously in the context of the present invention by varying the effects obtained by the light deviation means and/or by the possible movements of the fiber (2) in the support (3).

In some embodiments, the support is made of a biologically neutral material, with variable thermal conductivity to limit undesirable effects or amplify the thermal effect such as burning at the puncture site, and is resistant to high temperatures. The material is preferably selected from stainless steel or aluminum.

### Light Guide/Fiber

The optical fiber used may be of a common type, for example made of silica and particularly of a type used for interventional fibroscopy, endovenous laser, surgery, etc. The light guide may be made of a material other than silica, depending on the wavelength of the light, particularly laser light.

The cost of an instrument according to the invention can be divided by five or ten relative to an instrument currently used for the treatment of varicose veins by laser light if it is manufactured on a large scale avoiding the high cost of the operating theater if it is carried out in an medical practice or treatment room, and also allowing a repeated procedure in the event of a first result being insufficient.

In some embodiments, the numerical aperture of the fiber is between 15° and 40°, preferably 20° and 35°, ideally 23° and 32°, for vascular applications. It can be increased by introducing several fibers into the same channel in order to modify the shape of the photon field. Thus, in some embodiments, the instrument comprises two light guides, preferentially two optical fibers, one of the guides carrying a means of divergent optical deviation, for example convex polishing, while the other carries a means of convergent optical deviation, for example concave polishing. In some embodiments, the same light guide (2) comprises a means of divergent optical deviation and a means of convergent optical deviation. In some embodiments, the light guide (2) comprises a plurality of different optical fibers, as in confocal microscopy for example, with different means of optical deviation from one fiber to another, the instrument being coupled to a multiplexer at the light source to perform photodiagnostics, in photofluorescence for example, thanks to the differential collection of signals from the different fibers. Advantageously, the surface of the optical field determines the sectioning or destructive effect on the tissue, particularly when the fiber aperture is between 10° and 30°, preferably between 15 and 25°, or on the contrary widened for photodiagnosis and tissue fluorescence revealing abnormal proteins in cancerology, particularly when the aperture is between 20° and 40°, preferably 25-35°.

Indeed, the angle of the bevel determines the surface area of the ellipse corresponding to the notion of “optical surface”. The numerical aperture determines the “active surface” (212) within this optical surface. The angle of the bevel of the needle determines the size of this active surface (212), which is increased resulting in “optical amplification”. Illustrative and non-limiting examples of such a numerical aperture and active surface (212) are shown in FIG. 7, FIG. 16 and FIG. 17. It can be seen in FIG. 7 that the active surface will depend on the distal bevel and, in FIG. 17, that the additional deviation means (313), by polishing both the support (3) and the fiber (2), will deform the active surface by widening it, thus achieving a double effect at the distal end of the instrument. The first polishing surface (310) at the beveled distal end of the support allows a first effect and the second polishing (313) at the apical end allows a second effect, thanks to the different angles of the two polishings providing different light field modifications.

Numerical aperture is understood to mean the sine of the maximum input angle of the light into the light guide, for example the fiber, so that the light can be guided without loss, measured with respect to the fiber axis.

In some embodiments, the light guide is an optical fiber reflecting wavelengths between 200 nm and 5 μm. This allows the clinical applications to be broadened by targeting new chromophores. The fiber type can be selected with a wavelength window wider than infrared (250 nm, UV silica), such as 3 or 4 μm (Chalcogenides) for the needle and the light vector thereof. This allows clinical applications to be broadened by targeting new chromophores or fluorophores. Preferably, the fiber is made of silica.

Thus, the composition of the optical fiber will be selected based on the wavelengths used. The table below summarizes the different compositions of the guide according to wavelength.

| Guide composition | Wavelength (in μm) |
|---|---|
| Standard silica | 0.4-1.5 |
| UV silica | 0.23-1.5 |
| IR silica | 0.4-2.2 |
| | (Erbium, Holmium) |
| Sapphire | 0.4-3.3 |
| Fluoride glass | 0.4-5.5 |
| Chalcogenides | 5.5-11 |
| Texglass T.ox | 1-11 and 3-13 |
| AgCl and AgBr | 0.4-11 |
| Optical arms and hollow fibers | 10.6 (CO2) |

In some embodiments, the support is hollow and does not comprise fiber. The light guide then consists of a coating on the inner surface of the support forming an internal reflective tube such as those used in CO2 laser handpieces.

Advantageously, the tube thus allows long wavelengths, for example, 10600 nm (10.6 μm) to be reflected when a CO2 laser is used, rather than a conventional laser light source. As this wavelength is not very penetrating (20 μm deep), it is widely used for tissue detersion, for example in dermatological surgery or neurosurgery. Furthermore, this wavelength is not accessible to any other type of fiber and some embodiments of the present application therefore allow the use of such lasers, via a hollow fiber or a coating inside the needle.

In some embodiments, the CO2 laser can be conveyed by a hollow fiber which must stop just before the bevel. In fact, polishing the bevel is likely to result in the production of a powder residue that may render secondary light transmission incompatible. Note that FIG. 9 showing an illustrative and non-limiting example of such a hollow fiber, no means of light deviation is shown apart from the distal bevel, but it is of course possible to provide a means of deviation such as one of those described in the present application, as for example shown in FIG. 18, but also with any other embodiment featuring a means of deviation. In the field of CO2 lasers, tissue detersion plays a very important role in operating theaters, regardless of the specialty concerned (neurosurgery, ophthalmology, dermatology, vascular surgery, tumor surgery, gynecological surgery, etc.). Perfectly usable hollow fibers (2) can be integrated upstream of the bevel of the needle, whose correctly polished (metallic) end is quite sufficient to transmit this light and even focus it in order to optimize its sectioning and destructive effect on the tissue concerned.

In some embodiments, at least one colored filter is arranged in the light path. For example, the instrument comprises at least one filter and/or colorization to allow the use of a filtered and/or colorized white light, which is less expensive than a laser source. Thus, for example, at least a portion of the light guide (2) is filtered and/or colorized. This advantageously makes it possible to filter white light as a non-laser source (for example, PDT, tissue destruction, etc.), which is a source of savings, as lasers are more expensive than a filtered white light source (such as a flash lamp), and facilitates the use of certain processes such as those of dynamic phototherapy for example.

In some embodiments, the light guide comprises a doped optical fiber, preferentially with an outer diameter between 50 and 1000 μm, preferably between 50 and 150 μm, preferably between 75 and 125 μm. Advantageously, the doped fiber makes it possible to work on small-caliber fibers of the order of 100 μm in diameter for aesthetic applications particularly vascular photocoagulation and lipolysis, for example on the face. Preferably, the fiber is doped with erbium, holmium, thulium or praseodymium.

### Connector

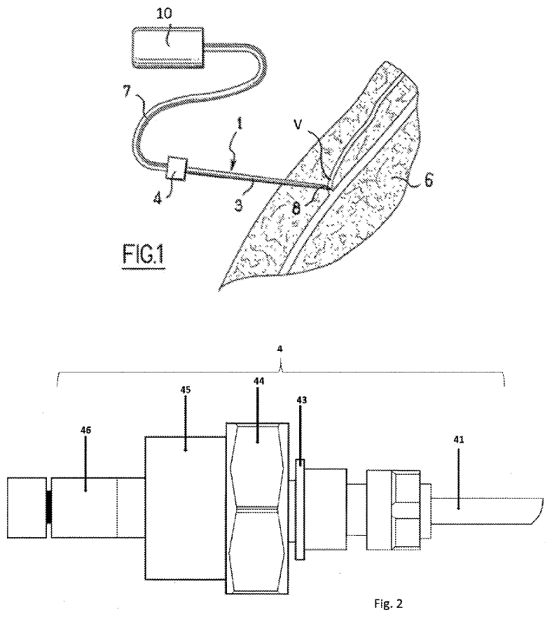

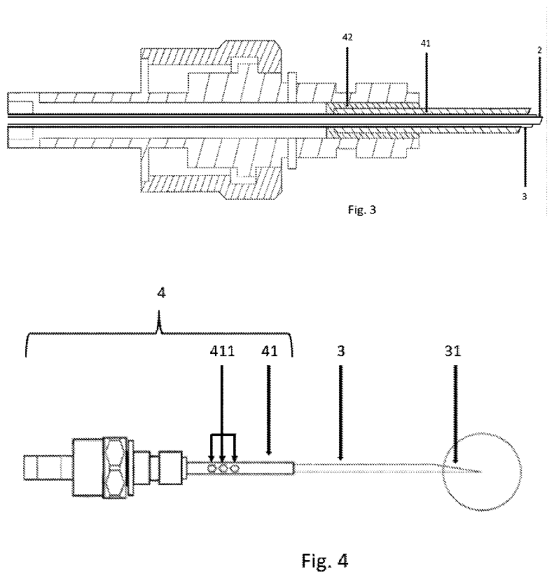

As already mentioned above, the instrument is preferably connected to the light source (10) by a connector (4) allowing the instrument to be changed, for example between two patients or two applications. This connector can be of the SMA type as known in the field and shown in FIG. 2, FIG. 3, FIG. 4 and FIG. 10, but it can be replaced by a locking clip connector, avoiding the use of a screw ferrule which could lead to incorrect positioning or breakage of the constituent components of the instrument. The present application therefore uses the terms “connector” or “fitting” or “connection” interchangeably, with or without the term SMA, but they are not limiting. In some embodiments, the needle and vector are connected inside a hollow metal guide, with the male connector of the needle fitting into the female connector of the light vector, preferably with spring-loaded balls to maintain the connector in the correct position for optical guidance.

FIG. 2 is a non-limiting example of some embodiments, wherein the fitting or connection means (4) comprises an SMA connector with a support and a screw ferrule. A mounting ring secures and stiffens the connection. A connecting tube (41) can be crimped in the connection means (4), and a reinforcing support (42) can be provided to further stiffen the connection. The support (3) is inserted into the connection tube (before or after connection). In some embodiments, as for example shown in a non-limiting manner in FIG. 4, the support (3) is a needle crimped in the crimping tube (41) which comprises one or more crimping points (411). In these embodiments, crimping is essential to ensure the rigidity of the needle and avoid any mechanical torsion or angulation that could alter the fiber inside the body of the needle (3). It is integrated in the periphery of this latter in the crimping body part of the connection means (4). Advantageously, the crimping also provides a mass reserve to contain excess heat generated by the needle in the tissue. Its composition, particularly its density influences parameters such as thermal conductivity, heat transfer, thermal inertia and thermal resistance. It is a buffer for excess heat which is a safety feature when using this equipment in vivo.

In some embodiments, the base of the support is crimped onto the connection clip. This improves strength to prevent any fiber breakage at the tip of the needle which can be a source of breakage and therefore accidents. The clip is upstream of the crimping and may be metal to increase the strength. In some embodiments, the base of the needle and connector features a bevel marker for the needle once it has been screwed down to maximum tightness or a clip with a facing to ensure that the bevel is correctly positioned. Some manipulations require the equipment to be rotated particularly in the vascular field.

In some embodiments, the light guide has a smaller diameter than that of the support, at the connection between the support and the light guide. Advantageously, this prevents overheating between the SMA connector of the needle and the safety factor light vector.

In some embodiments, a thermal reagent is included in the sheath of the light vector and the SMA clip or connector. This reagent may be non-metallic and single-use. Advantageously, this allows abnormal overheating to be visualized on the device.

In some embodiments, the instrument comprises means for rotating and/or advancing the light guide (2) and/or the support (3), particularly relative to each other. In some embodiments, the instrument comprises at least one marking to check the correct positioning of the instrument.

The present invention also relates to improving the ergonomics and use of a medical instrument. Although presented independently, and forming part of embodiments independent of the embodiments described above, it is readily understood by the person skilled in the art that the features described hereinafter can also be combined with what is described in the remainder of this text.

Thus, in some embodiments, the support (3), for example an optical needle, could be connected to a laser source of variable wavelength by a light guide (light vector) connected on either side to the laser and to the support by a connecting clip instead of a standard connector (SMA or other). This saves time and improves ergonomics by making it easier to hold. Additionally, screwing on an SMA connector can sometimes destroy the thread of the light guide (light vector) and the support itself, for example the needle itself. Additionally, the connection clip can be color-coded for each equipment depending on the application, making it even easier to use.

In some embodiments, the connection between the needle and the light vector is colorized. Depending on the application, this makes it easier to identify each type of equipment according to the application for which it is intended, preferably in the same color as the clip.

### Applications

A laser light treatment process can advantageously use an instrument or system according to the invention. It can be notably used for vascular applications, particularly for the treatment of varicose veins. Laser treatment can be used for sclerotherapy. It can also be used for arterial occlusion replacing chemical embolization and for fibro-calcareous arterial debulking in preparation for angioplasty. The same applies to ulcer detersion, tumor sectioning or laser lipolysis.

For example, after a local anesthetic, the instrument according to the invention is used to prick the varicose vein to be treated, through the skin, in the same way as a puncture. The injection can be performed under visual or ultrasound control. The instrument itself can be used to inject anesthetic through a dedicated channel, thus avoiding the need for an additional injection and syringe. A channel, possibly the same one, can also be provided to check blood flow. It is thus possible to check that the instrument is correctly positioned in the vascular system. Perivascular or peritumoral intumescence anesthesia may be used. Identification can be done under ultrasound. A photochemical substance can also be injected to potentiate the thermal or phototherapeutic effect if applied to tumor pathology.

Once the instrument is in position, a laser shot is fired. It is advantageous to rotate the end of the light guide to ensure circumferential lesioning of the wall of the varicose vein to be treated. The result is thus sclerosis of the varicose vein, which is no longer irrigated. Endovenous laser treatment produces photocoagulation of the blood with tissue retraction due to the contraction of collagen which is in greater proportion in the varicose vein.

The instrument is then removed, and can be decarbonized if necessary with a sterile compress. The instrument is then ready to treat another varicose vein. Direct puncture without a guiding needle is also possible.

In some embodiments, the instrument comprises an electronic accounting device (chip) with the light guide and source in order to calibrate the beam at the output of the support (needle outlet); this would enable the power loss at the tip of the needle to be accurately known compared with the data from the light source. Additionally, it could be coupled to a device enabling spectrophotometry or optical cell biopsy.

In some embodiments, the instrument comprises a thermocouple device at the distal end of the support to measure the temperature at this point, to provide an in vivo temperature measurement via an already patented double channel and an alarm system in the event of a critical temperature that prevents burns and allows in vivo and in vitro thermodynamic models to be studied with external thermography coupling for each application. Together, they can be used to design thermodynamic simulations. In addition, this would enable chromophores to be detected for screening toxic products in food control as soon as they have a specific absorption spectrum, thus avoiding slow and costly biological analyses and significantly increasing screening in the food chain.

The invention thus enables the detection of toxic products in food control using a medical instrument according to the invention, by chromophore detection.

The temperature study using a thermocouple based on the graph to be studied would make it possible to evaluate the percentage of fat and protein in ground meat in a food chain for example or study a tumor in situ in human or veterinary pathology.

In some embodiments, the use of a “multiplexer” type device allows emission at a certain wavelength and recovery of a frequency shift, particularly for immunofluorescence by fluorophore injection, for example via the needle channel or even the use of chromophore. Thus, some embodiments relate to the use of the instrument for detecting chromophores. In addition, this type of tool allows spectrometric analysis, particularly infrared, for example, to study tissues in vivo. This means that the tool can be used in medical research as well as in the veterinary and food sectors in order to search for specific substances in spectrophotometric analysis. For fluorescence, it may be useful for calibration to use an end whose tip allows this fluorescence to be tested.

Wavelength division multiplexing or WDM requires a low number of channels to be recruited for the needle and therefore remains less costly or “coarse WDM”. It uses a dichroic filter deposited on the fiber itself. It splits the photon field in two allowing bi-directional transmission to observe an absorption shift in spectrophotometry (for example, immunofluorescence) for tumor photodiagnosis, revealing abnormal proteins.

It is conceivable to multiply the number of fibers in a needle, even in a gage of just 2 mm, in order to exploit optical imaging equivalent to the “phased array” in ultrasound—reference is then made to “dense WDM” or “ultra-dense WDM”.

The present invention is therefore applicable to photodiagnostics when using a multiplexer at the light source, because with these multi-fiber systems, by modifying the optical field using the arrangements of the present application, particular images can be obtained with varying levels of specificity and sensitivity, particularly in immunofluorescence. Light is injected and its return is examined, but if a dual-channel needle is used (with an injection channel for immunofluorescent molecules), it is possible for example to discriminate cell types (astrocytoma versus glioma, for example) by recovering the frequency shift. Various dual-channel (or with 2 channels in 2 instruments) embodiments will therefore use one channel for injecting immunofluorescent molecules and the other for light, with the principles described in the present application.

Spectrometry (with an upstream multiplexer) can also be used to detect particular molecules (with specific absorption spectra). In this case, there is no need for two channels or an injection channel.

Applications include taking advantage of gradients or index jumps, particularly to convey several different wavelengths. There are essentially two types of optical fiber that exploit the principle of total internal reflection: the index hopping fiber and the graded-index fiber. In the index hopping fiber, the refractive index drops sharply from a value in the core to a lower value in the cladding. In the graded-index fiber, this index change is much more gradual. A third type of optical fiber uses the band gap principle of photonic crystals to guide light, rather than total internal reflection. Such fibers are called photonic crystal fibers, or micro-structured fibers. These fibers usually have a much higher index contrast between the different materials (usually silica and air). Under these conditions, the physical properties of the guide differ significantly from those of index hopping and graded index fibers. The invention can therefore cover the use of these various types of fiber. On the other hand, the invention can also be applied to single-mode and/or multimode fibers. Finally, the invention can also use dichroic filters.

In some embodiments, the wavelength of the light is between 200 and 5000 nm. For example, for arterial or venous unblocking, the wavelength may be around 308-310 nm.

As mentioned above, the numerical aperture of the fiber determines the active surface of the device that can be exploited for these various applications such as optical imaging, so-called optical biopsy, medical applications in the thermal field and also disruptive applications, notably in ophthalmology, arterial unblocking (stenosis or occlusion), and venous unblocking in the case of stenosis or occlusion. This can be used, for example, to prepare vessels for stenting, either arterial or venous. Vascular unblocking exploits wavelengths in the ultraviolet range as for industrial marking or industrial sectioning of materials. Since mission times are much shorter, peak powers are very high which means that the thermal effect at the biological level evolves towards a thermo-mechanical effect and tissue disruption which is a non-thermal effect and therefore safe for medical applications. On the other hand, as mentioned above, the source can be a white light filtered by the fiber itself to determine the correct wavelength for photodynamic therapy and photodiagnostics (PDT=Photo Dynamic Therapy), for example, with long emission times, that is, generally in excess of one second.

As mentioned above, in some embodiments, as for example shown in a non-limiting manner in FIG. 8, the optical needle is not polished into an arrow shape, and has no lateral polishing, allowing the needle to be transformed into an optical sectioning tool. This relates to the fields of tissue or tumor detersion, whether in the surgical field or medical practice procedures such as laser conization for stages I and II which are not treated in the operating room because of the cost. In this application, the tool can therefore be used to treat a small precancerous lesion in a medical practice with local anesthetic and a long needle thereby statistically improving prognosis. If treatment is deemed insufficient after the first histological biopsy, this treatment can be completed or repeated outside the operating theater always at low cost. The same applies to dermatological surgery for example (removal of ulcers, removal of small benign tumors under local anesthetic).

The laser light source requires modest power for this application, for example of the order of 10 to 15 W in the field of small portable lasers.

Thus, particularly for greater precision, it may be advantageous for the light guide, for example an optical fiber, to extend beyond the support, for example a needle. The fiber can be expected to protrude from 0 to 1 cm, preferably about 5 mm. To protect the optical fiber during puncture, means may be provided to retract the fiber into the needle and means to extend the optical fiber beyond the distal end of the needle up to a working position.

Means may also be provided for rotating the light guide relative to other parts of the instrument; for example, relative to the support, particularly in the case of a needle containing an optical fiber, or relative to connection means, particularly in the case of a needle internally clad with silica. These rotation means thus allow easy circumferential treatment.

An instrument according to the invention can also be used for other vascular applications, for example sclerosis of varicose veins not only in the lower limbs, but also pelvic or esophageal varicose veins (intraoperatively), hemorrhoids, or occlusion with vascular sclerosis or vascular anastomosis during surgery. An instrument according to the invention can also be used for tissue perforation, or as an alternative to biological adhesives, such as cyanoacrylates, for any intravascular embolization, in particular for congenital or acquired fistulas, or venous embolization, in particular for malformations or angiomas, or lymphatic occlusion causing ulcers.

Other applications include tissue destruction, particularly if high precision is required. Thus, in dermatology, the use of a laser with a wavelength of 980 nm, or the water-absorbed wavelengths of Erbiums and Holmiums, 2940 and 2140 μm, makes an instrument according to the invention particularly effective, especially for the treatment of condylomas, warts and other skin lesions. Scars are of superior quality to those obtained using other techniques.

In the context of cell destruction, other applications may also include contact-mode tissue detersion, for example applied to ulcers. An instrument according to the invention can also be used for the destruction of tissue metastases, for example intra-hepatic, which is more ergonomic and less costly than radiofrequency. Tissue destruction by laser involves the absorption of photons into the tissue with secondary conversion into heat. As this absorption is targeted at certain tissue-specific molecular constituents (chromophores), the thermomechanical effect is more specific than other thermal tissue heating processes such as radiofrequency for example (thermal effect). This specificity opens the way to new laser applications, such as photodynamic therapy in oncology for example.

The use of a rigid support for the light guide allows more precise operation and superior handling. Thus, the lesions caused by the treatment are more precise. Biological stimulation improves healing. Hemorrhage is reduced, as is the risk of infection, since carbonization involves temperatures of 350° C. or 400° C. or more, up to tissue incandescence at around 600° C. Post-operative pain is also reduced, particularly in the case of ulcer detersions; there are fewer post-operative infections. Secondary inhibition of inflammation and algogenic factors at nerve endings exists.

Additionally, parameters can be set, for example wavelength, emission and exposure time between each shot, fluence, irradiance, continuous or pulsed mode (single or multiple) or the destruction area according to optical scattering at the end of the light guide.

It will be readily understood upon reading the present application that the features of the present invention, as generally described and shown in the figures, can be arranged and designed in a wide variety of different configurations. Thus, the description of the present invention and the accompanying figures are not intended to limit the scope of the invention but simply represent selected embodiments.

The skilled person will understand that the technical features of a given embodiment can in fact be combined with features of another embodiment unless the opposite is explicitly stated or it is evident that these features are incompatible. Furthermore, the technical features described in a given embodiment may be considered separately from the other features of that embodiment unless explicitly stated otherwise.

It should be obvious to those skilled in the art that the present invention allows embodiments in many other specific forms without departing from the field defined by the scope of the appended claims; these should be considered by way of illustration and the invention should not be limited to the details given above.

### LIST OF REFERENCE SIGNS

1. Instrument
2. Light guide (optical fiber, hollow fiber or reflective coating)
21. Distal end of the light guide
210. Polishing area of the distal end of the light guide
212. Active surface of the light guide. Tubular support (or needle)
31. Beveled distal end of the support
310. Polishing area of the distal end of the support
311. Support orifice
312. Lateral orifice of the support
3121. Chamfer (or polishing) of the lateral orifice of the support
313. Second bevel or polishing of the support (either lateral, or central)
314. Distal indentations of the support orifice (lateral or central, semi-spherical or triangular)
315. Reflective polished inner surface of the support
4. Means of connection
41. Connection or crimping tube
411. Crimping points
42. Connecting tube support
43. Mounting ring
44. SMA lock (screw ferrule)
45. SMA connector support
46. SMA connector
6. Tissue or organ
7. Optical cable
8. Distal end of instrument
10. Light source
V. Blood vessel
H. Distal polishing height of the support and guide
B. Arrow angle of the distal end of the support
C. Polishing angle of the second lateral bevel of the distal end
G. Polishing angle of the second central bevel of the distal end
D. Bevel angle of the beveled distal end of the support
E. Angle of curvature of the support
F. Chamfer angle of the lateral orifice of the support.

1. A medical instrument comprising a free distal end intended to penetrate into a tissue or an organ and/or a blood vessel (V) and a proximal end connectable to a light source, said instrument comprising a tubular support in which is mounted at least one light guide, said support comprising a beveled distal end comprising at least one orifice forming a first optical outlet, for a substantially frontal emission of the light by said guide via said first optical output, said instrument further comprising at least one optical deflection means of the light forming a second optical output, for deflected light emission, comprising at least one machining on a distal portion of the instrument, with at least one reflective and/or concave polish configured for light convergence and one reflective and/or convex polish configured for a divergence of light, said polishing being carried out on said light guide and/or on said support.

2. The medical instrument according to claim 1, wherein the light guide comprises an optical fiber.

3. The medical instrument according to claim 2, wherein said optical fiber is hollow and associated with a CO2 laser light source.

4. The medical instrument according to claim 1, wherein the light guide comprises a reflective coating on the inner wall of the support tubular.

5. The medical instrument according to claim 1, wherein said beveled distal end of said support is sharpened with an arrow with a sharpening angle (B) giving it cutting and penetrating properties.

6. The medical instrument according to claim 1, wherein at least one distal portion is curved along a radius (E) of curvature, facilitating manipulation of the instrument from outside the tissue or organ.

7. The medical instrument according to claim 1, wherein said light deflecting means comprises at least one lateral orifice in the wall of said support, close to the distal end, said orifice lateral comprising at least one chamfer in the thickness of the wall of the support, providing an angle (F) of optical deflection, either by flaring outwards to form a deflection means diverging, or by widening inwards to form a converging means of deflection.

8. The medical instrument according to claim 1, wherein said light deflection means is a divergent deflection means comprising a convex polishing performed on a light-conducting ball deposited on the surface of the light guide.

9. The medical instrument according to claim 1, in which the distal end of the light guide, for example a transversely sectioned or beveled bare-tip optical fiber or a hollow fiber, is positioned recessed from the beveled distal end of the support, with a reflective polished internal surface inside the beveled distal end of the support then serving as a reflective tool forming an optical deflection means.

10. The medical instrument according to claim 1, wherein the beveled distal end of the support comprises a polishing area circumferential on the surface of the bevel at an angle (A) bevel and includes a second bevel, obtained by a second polishing of a portion of said polishing area.

11. The medical instrument according to claim 10, wherein said second polishing is carried out on a lateral portion of said polishing area, providing an angle (C) of lateral optical deviation.

12. The medical instrument according to claim 10, wherein said second polishing is circumferential over all of said polishing area and provides an angle (D) of distal optical deviation.

13. The medical instrument according to claim 10, in which said second polishing is carried out on a central and apical portion of said polishing area, providing an angle (G) of apical optical deviation.

14. The medical instrument according to claim 13, wherein said second polishing is carried out at a height (H1, H2) determined to also obtain an area (210) for polishing the distal end of the guide of light, resulting in two angles (G) of optical deviation and an active surface deformed from the guide of light.

15. The medical instrument according to claim 1, wherein the beveled distal end of the support comprises a polishing area circumferential on the surface of the bevel at an angle (A) beveling and comprises at least one semi-spherical and/or triangular notch forming at least one optical deflection means, either lateral or central and apical.

16. The medical instrument according to claim 1, wherein the distal end of the light guide is housed inside the support, set back relative to the distal end from the support by a distance of at least 1 to 10 mm.

17. The medical instrument according to claim 1, wherein the instrument comprises two light guides, preferably two optical fibers, one of the guides carrying a divergent optical deflection means, for example a convex polishing while the other carries a converging optical deflection means, for example a concave polishing.

18. The medical instrument according to claim 1, wherein said light guide comprises both a divergent optical deflection means and a convergent optical deflection means.

19. The medical instrument according to claim 1, in which the support comprises both a divergent optical deflection means and a convergent optical deflection means.

20. The medical instrument according to claim 1, wherein the instrument further comprises means for rotating and/or advancing the light guide and/or the support, in particular relative to each other.

21. The medical instrument according to claim 1, wherein the instrument comprises at least one marking to verify the correct positioning of the instrument.

22. The medical instrument according to claim 1, in which at least the colored filter is arranged in the light path.

23. The medical instrument according to claim 1, in which the support is made of a biologically neutral material, preferably chosen from stainless steel or aluminum.

24. The medical instrument according to claim 1, wherein the support has a distal outer coating (“coating”) of material with increased thermal conduction.

25. The medical instrument according to claim 1, in which the light guide is an optical fiber reflecting wavelengths between 200 nm and 5 pm.

26. The medical instrument according to claim 1, in which the light guide is a coating of the internal surface of the support forming an internal reflective tube reflecting wavelengths between 200 nm and 11 pm, preferably 5 pm and 11 pm.

27. The medical instrument according to claim 1, in which the light guide is a doped optical fiber with an outside diameter of between 50 and 1000 μm, preferably between 50 and 150 μm, preferably between 75 and 125 pm.

28. The medical instrument according to claim 1, in which the support has a length of between 10 and 120 mm, the outer diameter of the guide is between 100 and 4000 μm, preferably 50 and 1000 μm, the outside diameter of the needle is between 200 and 5000 μm, preferably between 450 μm and 1000 μm and the distal end of the support is beveled with a bevel angle (A) of between 10° and 20°.

29. The medical instrument according to claim 1, in which the instrument comprises an electronic accounting device with the light vector and the source in order to calibrate the power of the beam at the needle exit.

30. The medical instrument according to claim 1, in which the opening of the guide, preferably the numerical aperture of the fiber, is between 15° and 40°, preferably 20 and 35°, again preferably 23° and 32°.

31. The medical instrument according to claim 1, in which the instrument comprises two channels, either by a double channel in the support, or by a channel of the support associated with another channel of another instrument, the assembly being coupled to a thermocouple measuring device for measuring the temperature at the distal end of the support.

32. The medical instrument according to claim 1, which further comprises an alarm system when the value of the measured temperature exceeds a predetermined threshold value.

33. The medical instrument according to claim 1, which further comprises a thermal reagent configured to react when the latter reaches a predetermined temperature, in order to visualize overheating.

34. The medical instrument according to claim 1, in which the light guide comprises a plurality of different optical fibers, with different optical deflection means from one fiber to another, the instrument being coupled to a multiplexer at the level of the light source to carry out a photo-diagnosis thanks to the differential collection of the signals of the different fibers.

35. A method for detecting toxic products in food control by the use of a medical instrument according to claim 1, by the detection of chromophores.
